(19) United States
(12) Patent Application Publication
(10) Pub. No.: US 2024/0285771 A1
(43) Pub. Date: Aug. 29, 2024

(54) RAPIDLY DISINTEGRATING ORAL THIN-FILMS/FOAMS HAVING A HIGH ACTIVE-INGREDIENT CONTENT BASED ON A MIXTURE OF POLYVINYL ALCOHOLS HAVING VARIOUS MOLECULAR WEIGHTS

(71) Applicant: LTS Lohmann Therapie-Systeme AG, Andernach (DE)

(72) Inventors: Frank SEIBERTZ, Bad Breisig (DE); Marius BAUER, Andernach (DE); Marlene FUHRMANN, Kail (DE); Michael LINN, Waldböckelheim (DE); Marco EMGENBROICH, Rheinbach (DE)

(21) Appl. No.: 18/692,408

(22) PCT Filed: Sep. 6, 2022
(86) PCT No.: PCT/EP2022/074724

§ 371 Date: Mar. 15, 2024

(30) Foreign Application Priority Data
Sep. 17, 2021 (EP) 21197405.0

(51) Int. Cl. A61K 47/32; A61K 9/00; A61K 31/135; A61K 31/57; A61K 47/10; A61K 47/26

(52) U.S. Cl. CPC A61K 47/32; A61K 9/006; A61K 31/135; A61K 31/57; A61K 47/10; A61K 47/26

(57) ABSTRACT

The invention relates to an oral thin film comprising at least one matrix layer, wherein the at least one matrix layer comprises at least one polyvinyl alcohol having a low average molecular weight and at least one polyvinyl alcohol having a high average molecular weight and at least one pharmaceutical active substance. The invention also relates to the use of the oral thin film as a medicine.

The present invention relates to an oral thin film containing at least one active pharmaceutical ingredient and to the use of such an oral thin film as a drug.

Oral thin films (OTFs) are thin, flexible films based on a polymer matrix and loaded with active ingredients for drug delivery. The oral thin films are taken orally and dissolve immediately in the mouth or are applied to the mucosa. They are placed on or under the tongue or buccally, where they dissolve or disintegrate.

Oral thin films can be formed as non-foamed films or as foamed films or foam. OTFs are sometimes also referred to as oral thin films.

EP 422100 B1 discloses the active ingredient ulipristal acetate. Ulipristal acetate is a well-known emergency contraceptive (“morning-after pill”). It is administered as a tablet (“EllaOne®”) containing 30 mg micronized ulipristal acetate, and as further ingredients lactose monohydrate, povidone, croscarmellose sodium, and magnesium stearate. EllaOne® was approved in the European Union in 2009.

Ketamine is another known active pharmaceutical ingredient that is used more particularly for the treatment or prevention of pain.

For the administration of some active pharmaceutical ingredients, high drug loadings of the oral thin film are desirable, e.g. oral thin films with ulipristal acetate as the active ingredient with 30 mg per single dose. However, the problem with such oral thin films or oral foams with high drug loading per single dose is that comparatively large amounts of polymer are required to produce a film or foam that has acceptable flexibility, so that brittle films are avoided. However, the high polymer content leads to an increase in the thickness or basis weight of the films or foams. As a result, these formulations exhibit increased disintegration times. This is undesirable for films or foams that are supposed to disintegrate very quickly in the mouth.

In the first foam formulations tested, which contained ulipristal acetate as the active ingredient and were based on polyvinyl alcohol (PVA) with low molecular weight (PVA 4-88, MW approx. 31,000 Da), the content of PVA was 63.9%. With this concentration, it was possible to produce an ulipristal acetate foam with good haptic properties (see formulation A1 in the experimental section). However, as described above, the high polymer concentration led to an increase of the basis weight (180 g/m²) and the disintegration time.

In addition, known oral thin films with high active ingredient loading have the disadvantage that the maximum basis weight and thus the amount of active pharmaceutical ingredient contained therein is determined by the drying of the oral thin film during its manufacture. The higher the basis weight of the oral thin film, the more active pharmaceutical ingredient it may contain, but this prolongs the drying time of the oral thin film to a time that is no longer economically acceptable and can also lead to an inhomogeneous distribution of the active ingredient in the oral thin film.

The object of the present invention is to remove the above-mentioned disadvantages. More particularly, the object of the present invention is to provide flexible and stable oral thin films/foams which allow high drug loading while having an acceptable film thickness and a rapid disintegration time on application.

This object was surprisingly achieved according to claim 1 by using a mixture of polyvinyl alcohols with different molecular weights in the matrix layer. An additional improvement was achieved by adding humectants, more particularly certain sugar alcohols or starch derivatives.

Accordingly, the invention relates to an oral thin film comprising at least one matrix layer, wherein the at least one matrix layer comprises at least one polyvinyl alcohol having a lower average molecular weight of 10,000 to about 75,000 g/mol, preferably 10,000 to about 40,000 g/mol, and at least one polyvinyl alcohol having a higher average molecular weight of 140,000 to 300,000 g/mol, preferably 140,000 to 220,000 g/mol, and at least one active pharmaceutical ingredient, wherein said at least one polyvinyl alcohol having a lower average molecular weight and said at least one polyvinyl alcohol having a higher average molecular weight are present in the matrix layer in a total amount of 15 to 70 wt %, preferably from 15 to 65 wt %, based on the total weight of the matrix layer, and the weight ratio of said at least one polyvinyl alcohol having a lower average molecular weight to said at least one polyvinyl alcohol having a higher average molecular weight is in the range from 20:1 to 2:1.

The independent claim relates to the use of the oral thin film according to the invention. Preferred embodiments are indicated in the dependent claims.

In the following, PVA is used as an abbreviation for polyvinyl alcohol. The polyvinyl alcohol with a lower average molecular weight of 10,000 to about 75,000 g/mol, preferably 10,000 to about 40,000 g/mol, used according to the invention is also referred to below as “low molecular weight polyvinyl alcohol” or “low molecular weight PVA”. The polyvinyl alcohol with a higher average molecular weight of 140,000 to 300,000 g/mol, preferably 140,000 to 220,000 g/mol, is also referred to below as “high molecular weight polyvinyl alcohol” or “high molecular weight PVA”. In the present specification, “comprising” can also mean “consisting of”.

By adding a relatively low concentration, e.g. 6 wt %, of high molecular weight PVA, e.g. PVA 40-88, to a low molecular weight PVA, it was surprisingly possible to significantly reduce the total polymer concentration in the formulation (in the example, from 65.8% (formulation A1) to 39% (formulation A2)) without losing the dimensional stability of the previous formulations and without noticeably increasing the disintegration time. Since the PVA mixture according to the invention, e.g. PVA 4-88/40-88, allows the use of a lower total polymer concentration, it was also possible to reduce the basis weight of the oral films significantly, e.g. from 180 to 109.5 g/m². This improved the disintegration time compared to formulations containing only low molecular weight PVA.

Without wishing to be bound by any theory, it is assumed that the longer-chain PVA, such as PVA 40-88, in this mixture represents a kind of structure-forming agent that enables the incorporation of lower PVA concentrations.

The oral thin film according to the invention has at least one matrix layer. The at least one matrix layer comprises at least one polyvinyl alcohol having a lower average molecular weight in the range from 10,000 to about 75,000 g/mol, preferably 10,000 to 40,000 g/mol, more preferably 25,000 to 35,000 g/mol, and at least one polyvinyl alcohol having a higher average molecular weight in the range from 140,000 to 300,000 g/mol, preferably 140,000 to 220,000 g/mol, more preferably 200,000 to 210,000 g/mol.

Unless otherwise stated, all average molecular weights of polymers mentioned refer to the weight average molecular weight (Mw) determined by gel permeation chromatography.

In order to produce PVA, polyvinyl acetate is usually produced first, the acetate groups of which are then hydrolyzed. Commercially available polyvinyl alcohols can have a degree of hydrolysis of 84 to 92 mol %, preferably 86 to 90 mol %, and thus still contain a residual content of acetyl groups; a degree of hydrolysis of 98 to 99 or 86 to 89 mol % are common.

According to the present invention, a combination of at least one polyvinyl alcohol having an average molecular weight of about 31,000 g/mol and at least one polyvinyl alcohol having an average molecular weight of about 205,000 g/mol is particularly suitable.

In a preferred embodiment, the oral thin film therefore comprises a matrix layer in which the at least one polyvinyl alcohol having a lower average molecular weight is polyvinyl alcohol 4-88 or polyvinyl alcohol 8-88 and/or the at least one polyvinyl alcohol having a higher average molecular weight is polyvinyl alcohol 40-88. These polyvinyl alcohols are commercially available. If the molecular weight of the polyvinyl alcohol with a higher average molecular weight is too high, the resulting mixture is too viscous, which makes processing difficult or almost impossible. If the molecular weight is too low, no sufficient structuring of the resulting mixture will be achieved.

The total amount of the at least one polyvinyl alcohol with a lower average molecular weight and of the at least one polyvinyl alcohol with a higher average molecular weight in the matrix layer is in the range from 15 to 65 wt %, preferably from 20 to 60 wt %, more preferably from 22 to 58 wt %, more particularly from 24 to 56 wt % or from 22 to 50 wt % or from 25 to 40 wt %, based on the total weight of the matrix layer.

The weight ratio (A:B) of the at least one polyvinyl alcohol with a lower average molecular weight (A) to the at least one polyvinyl alcohol with a higher average molecular weight (B) is in the range from 20:1 to 2:1, preferably from 15:1 to 3:1, more preferably from 12:1 to 4:1, or from 10:1 to 4:1 or from 10:1 to 2:1, more particularly from 9:1 to 7:1.

The amount of the at least one polyvinyl alcohol with a higher average molecular weight in the matrix layer can be relatively small, e.g. in the range from 1 to 18 wt % or from 1 to 16 wt % or from 1 to 12 wt %, preferably from 2 to 10 wt %, more particularly from 3 to 4 wt %, based on the total weight of the matrix layer.

It is preferred that the at least one polyvinyl alcohol with a lower average molecular weight is a polyvinyl alcohol with a lower average molecular weight and the at least one polyvinyl alcohol with a higher average molecular weight is a polyvinyl alcohol with a higher average molecular weight.

The polyvinyl alcohols used function as matrix formers for the matrix layer, which comprises a polymer matrix. It is preferred that no other polymers, more particularly no other matrix-forming polymers, are contained in the matrix layer in addition to these polyvinyl alcohols. If other polymers are included, the amount of polymers other than the polyvinyl alcohols mentioned is preferably not higher than 15 wt %, more preferably not higher than 10 wt %, based on the total weight of the matrix layer. Examples of other polymers that may be used, if applicable, include hydroxypropyl methyl cellulose (HPMC), polyvinylpyrrolidone (PVP), hydroxypropyl cellulose (HPC), polyethylene glycol (PEG) or mixtures of these polymers.

The matrix layer of the oral thin film according to the invention further comprises at least one active pharmaceutical ingredient, e.g. one active pharmaceutical ingredient or a combination of two or more active pharmaceutical ingredients. The at least one active pharmaceutical ingredient is in principle not subject to any limitation, but is preferably selected from all active pharmaceutical ingredients that can be administered orally and/or transmucosally. According to the present invention, the active pharmaceutical ingredient also subsumes, for example, all pharmaceutically acceptable salts and solvates of the respective active pharmaceutical ingredient.

The at least one active pharmaceutical ingredient may be selected, for example, from active pharmaceutical ingredients of the active ingredient classes of emergency contraceptives, analgesics, hormones, hypnotics, sedatives, antiepileptics, amphetamines, psychoneurotropic agents, neuromuscular-blocking agents, antispasmodics, antihistamines, antiallergics, cardiotonic agents, antiarrhythmics, diuretics, hypotensive agents, vasopressors, antidepressants, antitussives, expectorants, thyroid hormones, sex hormones, antidiabetics, antitumor agents, antibiotics, chemotherapeutics and narcotics, wherein the list is not exhaustive.

The at least one active pharmaceutical ingredient is preferably a particulate active pharmaceutical ingredient that is dispersed in the matrix or polymer matrix. The at least one active pharmaceutical ingredient may be a water-soluble active ingredient or an active ingredient that is sparingly soluble or insoluble in water. Herein, a water-soluble active ingredient refers to an active ingredient with a solubility in water of more than 10 g/L at a temperature of 20° C. Herein, a sparingly water-soluble or water-insoluble active ingredient refers to an active ingredient with a solubility in water of not more than 10.0 g/L at a temperature of 20° C. The specification is derived from the definitions in the European Pharmacopoeia.

The active ingredient may be present in the formulation for the preparation of the oral thin film in dissolved form, in suspended form or in both dissolved and suspended form.

The active ingredient can be present as a starting material in particle form. If the starting material of the active ingredient is particulate, it may be micronized particles or non-micronized particles. The particle size can range from 0.2 μm to 1 mm, for example.

In a preferred embodiment, the at least one active pharmaceutical ingredient is or comprises ulipristal acetate, preferably micronized ulipristal acetate. Ulipristal acetate is 17-acetoxy-11-(4-N,N-dimethylaminophenyl)-19-norpregna-4,9-diene-3,20-dione) with the following chemical formula:

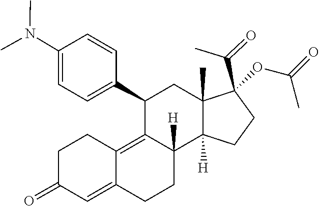

The active ingredient ulipristal acetate is dispersed in the polymer matrix of the matrix layer. In the matrix layer, ulipristal acetate is preferably present in solid form, more particularly in crystalline form, as particles.

The ulipristal acetate as starting material may be, for example, non-micronized or micronized ulipristal acetate. The particle size of the ulipristal acetate may be in the range from 0.2 μm to 1 mm, e.g. 0.2 to 100.0 μm or 0.5 to 40.0 μm.

The particle size refers to the volume-weighted particle size d90 and can be determined, for example, by laser diffraction analysis, dynamic light scattering or sieve analysis. The volume-weighted particle size d90 refers to the volume-weighted particle size in which 90% of the distribution has a smaller particle size and ten percent has a larger particle size, as is known to one skilled in the art.

In another preferred embodiment, the at least one active pharmaceutical ingredient comprises or is ketamine or a pharmaceutically acceptable salt thereof. Ketamine is understood here to mean (S)-(±)-2-(2-chlorophenyl)-2-(methylamino)cyclohexan-1-one, (R)-(±)-2-(2-chlorophenyl)-2-(methylamino)cyclohexan-1-one, and the racemate (RS)-(±)-2-(2-chlorophenyl)-2-(methylamino)cyclohexan-1-one. The ketamine preferably is (S)-ketamine or a pharmaceutically acceptable salt thereof, wherein ketamine is preferably present as an HCl salt, such as (S)-ketamine HCl, or as a free base. such as (S)-ketamine HCl. Ketamine is used as an analgesic, antidepressant, or anesthetic.

The ketamine as starting material may be present as micronized particles or as non-micronized particles. Suitable average particle sizes are, for example, in the range from 1 to 1000 μm or from 5 to 500 μm or from 10 to 200 μm. The particle size can be determined using an optical microscope, for example.

In another preferred embodiment, the at least one active pharmaceutical ingredient comprises or is naloxone, more particularly naloxone HCl dihydrate. Naloxone is an opioid antagonist and belongs to the group of pure opioid antagonists that act as competitive antagonists on all opioid receptors. Thus, they partially or completely cancel out the effects caused by opiates and opioids.

In another preferred embodiment, the at least one active pharmaceutical ingredient comprises or is nalmefene, more particularly nalmefene HCl. Nalmefene is an opioid antagonist and thus partially or completely reverses the effects caused by opiates and opioids. Nalmefene is also used in the treatment of alcoholism.

In another preferred embodiment, the at least one active pharmaceutical ingredient comprises or is vortioxetine, more particularly vortioxetine HBr. Vortioxetine is an antidepressant drug with a multimodal mechanism of action.

The at least one active pharmaceutical ingredient, or a pharmaceutically acceptable salt thereof, is preferably present in the matrix layer in an amount in the range from 10 to 60 wt %, preferably from 20 to 55 wt %, more preferably from 30 to 50 wt %, based on the total weight of the matrix layer.

In a particularly preferred embodiment, the matrix layer further includes at least one hygroscopic additive having more than 3 carbon atoms, which is preferably selected from sugars, sugar alcohols, starch and starch derivatives, or mixtures thereof.

The additional inclusion of a hygroscopic excipient, such as sugar, sugar alcohols, starch, and/or starch derivatives, leads to a further noticeable reduction in the disintegration time (see formulation A2 in the examples). Without wishing to be bound by a theory, it is assumed that the effect is based on the fact that the hygroscopic excipients quickly “draw” water into the formulation, which then leads to a faster disintegration time compared to a reference formulation without such a hygroscopic excipient due to the low polymer concentration.

Preferred specific examples of suitable hygroscopic excipients with more than 3 carbon atoms are mannitol, isomalt, lactitol, sorbitol (glucitol) and xylitol, threitol, erythritol, starches such as corn or rice starch, including starch derivatives, sucralose, or mixtures of two or more of these compounds. A particularly preferred hygroscopic additive is sorbitol. The hygroscopic additives can also be described here as disintegrants.

It is possible to incorporate one hygroscopic excipient or a combination of two or more. The at least one hygroscopic excipient having more than 3 carbon atoms, if used, may be present in the matrix layer, for example, in an amount of up to 20 wt %, preferably in an amount of 5 to 20 wt %, more preferably 5 to 15 wt %, based on the total weight of the matrix layer.

The matrix layer may further comprise at least one excipient, e.g. selected from the group consisting of colorants, flavorings, sweeteners, plasticizers, taste-masking agents, emulsifiers, enhancers, pH regulators, disintegrants, solubilizers, preservatives, anti-foaming agents (for film production only) and/or antioxidants. It is preferred that the matrix layer comprises one or more plasticizers, preferably glycerol.

Each of these excipients, if present, is preferably present in the matrix layer in an amount of 0.1 to 15 wt %, preferably 0.1 to 10 wt % or 0.1 to 5 wt %, based on the total weight of the matrix layer.

The matrix layer of the oral thin film may be a non-foamed matrix layer or a foamed matrix layer, wherein the matrix layer is preferably a foam. The configuration as a non-foamed matrix layer or as a foamed matrix layer is known to those skilled in the art.

In the case of a foamed matrix layer, the layer may be in the form of a solidified foam that has spaces or cavities filled with a gas, a gas mixture, a liquid, or a liquid mixture. Such OTFs are usually referred to as “foam OTFs”. The cavities in foam OTFs and the associated larger surface area of the films can accelerate the dissolution of the dosage form and the release of the active ingredient.

In addition to the production of foams, the components of the matrix layer according to the invention also enable the production of non-foamed OTFs with a high loading of active ingredient (e.g. 43%) and an acceptable disintegration time (see example of formulation A4). Without the addition of the high-molecular PVA, the maximum loading of active ingredient would have to be lower, which would have to be compensated for by increasing the basis weight. This in turn would lead to longer disintegration times. With non-foamed OTFs, in contrast to foams, there is the additional complication that the possible increase in basis weight is very limited. If a basis weight of more than 170 g/m² is reached, the drying process becomes very time-consuming and therefore unprofitable.

The basis weight of the matrix layer is preferably in the range of 50 to 220 g/m², more preferably 50 to 150 g/m², particularly preferably 70 to 130 g/m².

The matrix layer of the oral thin film according to the invention has, for example, a surface area in the range from 0.3 to 20 cm², preferably 1 to 10 cm². The thickness of the matrix layer can be in the range, for example, from 10 to 3000 μm, preferably from 90 μm to 2000 μm, more preferably from 40 to 400 μm.

In a preferred embodiment, the matrix layer comprises at least 1 mg, preferably at least 20 mg, more preferably at least 30 mg of active ingredient, e.g. 25 to 150 mg, preferably 30 to 150 mg, of active ingredient, more particularly ulipristal acetate, ketamine, naloxone, nalmefene or vortioxetine, more particularly naloxone HCl dihydrate or nalmefene HCL or vortioxetine HBr.

Particularly preferably, the weight ratio of the at least one polyvinyl alcohol having a lower average molecular weight to the at least one polyvinyl alcohol having a higher average molecular weight and the respective total amounts of the at least one polyvinyl alcohol having a lower average molecular weight and the at least one polyvinyl alcohol having a higher average molecular weight are adapted to an active ingredient contained in the oral thin film.

Thus, it is preferred that in an oral thin film, more particularly as defined above, in which ulipristal acetate or a pharmaceutically acceptable salt thereof is present as the active pharmaceutical ingredient, the weight ratio of the at least one polyvinyl alcohol having a lower average molecular weight to the at least one polyvinyl alcohol having a higher average molecular weight is in the range of 10:1 to 4:1. Additionally or independently, it is preferred that the amount of the at least one polyvinyl alcohol having a lower average molecular weight is in the range of 20 to 40 wt %, based on the total weight of the matrix layer. Additionally or independently, it is preferred that the amount of the at least one polyvinyl alcohol with a higher average molecular weight is 2 to 10 wt %, based on the total weight of the matrix layer. Additionally or independently, it is preferred that the amount of ulipristal acetate or the pharmaceutically acceptable salt thereof is 35 to 45 wt %, based on the total weight of the matrix layer.

Further, it is preferred that in an oral thin film, more particularly as defined above, in which ketamine or a pharmaceutically acceptable salt thereof is present as the active pharmaceutical ingredient, the weight ratio of the at least one polyvinyl alcohol having a lower average molecular weight to the at least one polyvinyl alcohol having a higher average molecular weight is in the range of 10:1 to 4:1. Additionally or independently, it is preferred that the amount of the at least one polyvinyl alcohol having a lower average molecular weight is in the range of 20 to 40 wt %, preferably of 25 to 35 wt %, based on the total weight of the matrix layer. Additionally or independently, it is preferred that the amount of the at least one polyvinyl alcohol with a higher average molecular weight is 2 to 10 wt %, based on the total weight of the matrix layer. Additionally or independently, it is preferred that the amount of ketamine or the pharmaceutically acceptable salt thereof is 35 to 45 wt %, based on the total weight of the matrix layer.

Further, it is preferred that in an oral thin film, more particularly as defined above, in which naloxone or a pharmaceutically acceptable salt thereof is present as the active pharmaceutical ingredient, the weight ratio of the at least one polyvinyl alcohol having a lower average molecular weight to the at least one polyvinyl alcohol having a higher average molecular weight is in the range of 10:1 to 2:1. Additionally or independently, it is preferred that the amount of the at least one polyvinyl alcohol having a lower average molecular weight is in the range of 45 to 60 wt %, preferably of 50 to 58 wt %, based on the total weight of the matrix layer.

Additionally or independently, it is preferred that the amount of the at least one polyvinyl alcohol with a higher average molecular weight is 5 to 20 wt %, based on the total weight of the matrix layer. Additionally or independently, it is preferred that the amount of naloxone or the pharmaceutically acceptable salt thereof is 5 to 15 wt %, preferably about 10 wt %, based on the total weight of the matrix layer.

Further, it is preferred that in an oral thin film, more particularly as defined above, in which nalmefene or a pharmaceutically acceptable salt thereof is present as the active pharmaceutical ingredient, the weight ratio of the at least one polyvinyl alcohol having a lower average molecular weight to the at least one polyvinyl alcohol having a higher average molecular weight is in the range of 10:1 to 2:1. Additionally or independently, it is preferred that the amount of the at least one polyvinyl alcohol having a lower average molecular weight is in the range of 45 to 60 wt %, preferably of 50 to 58 wt %, based on the total weight of the matrix layer. Additionally or independently, it is preferred that the amount of the at least one polyvinyl alcohol with a higher average molecular weight is 5 to 20 wt %, based on the total weight of the matrix layer. Additionally or independently, it is preferred that the amount of nalmefene or the pharmaceutically acceptable salt thereof is 5 to 15 wt %, preferably about 10 wt %, based on the total weight of the matrix layer.

Further, it is preferred that in an oral thin film, more particularly as defined above, in which vortioxetine or a pharmaceutically acceptable salt thereof is present as the active pharmaceutical ingredient, the weight ratio of the at least one polyvinyl alcohol having a lower average molecular weight to the at least one polyvinyl alcohol having a higher average molecular weight is in the range of 10:1 to 2:1. Additionally or independently, it is preferred that the amount of the at least one polyvinyl alcohol having a lower average molecular weight is in the range of 20 to 40 wt %, preferably of 30 to 40 wt %, based on the total weight of the matrix layer. Additionally or independently, it is preferred that the amount of the at least one polyvinyl alcohol with a higher average molecular weight is 2 to 12 wt %, based on the total weight of the matrix layer. Additionally or independently, it is preferred that the amount of vortioxetine or the pharmaceutically acceptable salt thereof is 25 to 35 wt %, based on the total weight of the matrix layer.

The oral thin film according to the invention is not subject to any limitations with regard to its structure. The following configurations are independent of whether the matrix layer is foamed or non-foamed.

The OTF according to the invention may be formed as a single-layer or multi-layer film, wherein a single-layer film is preferred. Thus, the oral thin film according to the invention may be present as a single-layer oral thin film and thus consist only of the matrix layer as defined above.

In another embodiment, the OTF may be present as a multilayer OTF and thus contain further layers in addition to the matrix layer as defined above. These additional layers may be laminated directly on top of each other or coated on top of each other or bonded with an adhesive layer in between. An adhesive layer is understood to be a layer that can act as an adhesive, as defined in DIN EN 923:2016-03. A non-adhesive layer can therefore not act as an adhesive as defined above.

Other layers can be, for example, buffer layers to adjust the pH, or slowly soluble or insoluble layers that protect the oral thin film from premature erosion. Alternatively, other matrix layers may be present that contain other active pharmaceutical ingredients or flavorings or flavor-masking ingredients. Other conceivable layers are adhesive layers and protective top layers.

The oral thin film according to the invention can be prepared by conventional methods known to those skilled in the art. Conventional methods for preparing the oral thin film according to the invention comprise, for example, the steps of:
a) preparing of a solution, dispersion or melt comprising the components of the matrix layer according to the invention, e.g. a dispersion in water or in a mixture of water and alcohol;
a1) optionally foaming the solution, dispersion or melt from step a) by introducing a gas or gas mixture, by chemical gas generation or by expansion of a dissolved gas or with the aid of a foaming machine;
b) spreading or pouring the solution, dispersion or melt from step a) or the optionally foamed solution, dispersion or melt from step a1) onto a surface or into a mold and cooling or drying.

The present invention further relates to an oral thin film as described above for use as a drug.

In a preferred embodiment, the oral thin film is used as an emergency contraceptive. In this case, the OTF contains an emergency contraceptive as the active pharmaceutical ingredient, preferably ulipristal acetate. The above details on preferred embodiments of ulipristal acetate evidently also apply to the use.

In another preferred embodiment, the oral thin film is used for the treatment or prevention of pain and/or depression or as an anesthetic. In this case, the OTF contains more particularly an analgesic, an anesthetic or an antidepressant as the active pharmaceutical ingredient, preferably ketamine or vortioxetine. The above details on preferred embodiments of ketamine or vortioxetine evidently also apply to the use.

In another preferred embodiment, the oral thin film is used in the treatment of opiate overdosing. In this case, the OTF contains more particularly an opioid antagonist as the active pharmaceutical ingredient, preferably naloxone, more particularly naloxone HCl dihydrate, or nalmefene, more particularly nalmefene HCl.

The invention is explained in more detail below with reference to non-limiting examples.

### EXAMPLES

### Example 1

For all of the following formulations in Example 1, the single-dose dosage forms resulting from them are meant to have a surface area of 7.04 cm² and are meant to contain, for the examples according to the invention, a single dose of at least 30 mg of ulipristal acetate or ketamine.

Formulation A1: Example of a PVA foam formulation without PVA mix and sugar alcohols

(reference formulation)

| | Content | |
|---|---|---|
| Ingredient | in [wt %] | Function of the ingredient |
| Ulipristal acetate | 23.7 | Drug |
| PVA 4-88 | 64.2 | Film polymer |
| Glycerol | 8.8 | Plasticizer |
| Saccharin Na | 2.2 | Sweetener |
| Sucralose | 1.1 | Sweetener, disintegrant, and humectant |
| Basis weight | 180 g/m² | |
| Process solvent | Water | |

Since the formulation only contains PVA 4-88, it can only be produced with a high polymer content of 64.2%, and therefore with a high basis weight. If the polymer content were lower, the film would be too brittle and, in the worst case, could no longer be made up. Additionally, the high basis weight has a negative effect on the disintegration time.

Formulation A2: Example of a PVA foam formulation with PVA Mix and sugar alcohols

| | Content | |
|---|---|---|
| Ingredient | in [wt %] | Function of the ingredient |
| Ulipristal acetate | 39.0 | Drug |
| PVA 4-88 | 31.0 | Film polymer |
| PVA 40-88 | 6.0 | Film polymer |
| Xylitol | 2.0 | Sweetener and disintegrant |
| Tween 80 | 1.0 | Plasticizer |
| Sorbitol | 9.0 | Sweetener and disintegrant |
| Sucralose | 1.0 | Sweetener, disintegrant, and humectant |
| Glycerol | 10.0 | Plasticizer |
| Menthol | 1 | Flavor |
| Basis weight | 109.5 g/m² | |
| Process solvent | Water | |

This formulation has a lower basis weight than the reference formulation. In addition, it has acceptable haptic properties and a significantly shorter disintegration time than the reference

Formulation A3: Example of a PVA foam formulation with PVA Mix without sugar alcohols

| | Content | |
|---|---|---|
| Ingredient | in [wt %] | Function of the ingredient |
| Ulipristal acetate | 39.0 | Drug |
| PVA 4-88 | 38.0 | Film polymer |
| PVA 40-88 | 8.0 | Film polymer |
| Saccharin Na | 2.0 | Sweetener |
| Tween 80 | 1.0 | Plasticizer |
| Sucralose | 1.0 | Sweetener, disintegrant, and humectant |
| Glycerol | 10.0 | Plasticizer |
| Menthol | 1.0 | Flavor |
| Basis weight | 109.5 g/m² | |
| Process solvent | Water | |

Omitting sorbitol increases the disintegration time of the foam (although still much faster than the reference). This shows that sugar alcohols or starch derivatives in the formulation improve disintegration. Due to their high hydrophilicity, sugar alcohols or starch derivatives draw water into the foam or film, which improves solubility.

Formulation A4: Example of a PVA film formulation with PVA Mix, sugar alcohols and rice starch

| | Content | |
|---|---|---|
| Ingredient | in [wt %] | Function of the ingredient |
| Ulipristal acetate | 43.0 | Drug |
| PVA 4-88 | 24.0 | Film polymer |
| PVA 40-88 | 3.0 | Film polymer |
| Span 85 | 1.0 | Plasticizer |
| Xylitol | 2.0 | Sweetener and disintegrant |
| Tween 80 | 1.0 | Plasticizer |
| Sorbitol | 9.0 | Sweetener and disintegrant |
| Sucralose | 1.0 | Sweetener, disintegrant, and humectant |
| Glycerol | 9.0 | Plasticizer |
| Menthol | 1.0 | Flavor |
| Rice starch | 6.0 | Disintegrant |
| Basis weight | 99.5 g/m² | |
| Process solvent | Water | |

The film has a high concentration of active ingredients and a low basis weight. Although it is a film, the formulation has a shorter disintegration time than the reference formulation.

Formulation B1: Example of a PVA film formulation with PVA Mix and sugar alcohols

| Starting materials | Composition [wt %] | Type/Function |
|---|---|---|
| Ketamine HCl | 38.0 | Active ingredient |
| PVA 4-88 | 29.0 | Polymer |
| PVA 40-88 | 3.0 | Polymer |
| Span 85 | 1.0 | Plasticizer |
| Xylitol | 2.0 | Sweetener and disintegrant |
| Tween 80 | 1.0 | Plasticizer |
| Sorbitol | 9.0 | Sweetener and disintegrant |
| Sucralose | 1.0 | Sweetener, disintegrant, and |
| | | humectant |
| Glycerol | 9.0 | Plasticizer |
| Menthol | 1.0 | Flavor |
| Rice starch | 6.0 | Disintegrant |
| Basis weight | 112.5 g/m² | |
| Solvent | Water | |

Formulation B2: Example of a PVA foam formulation with PVA Mix and sugar alcohols

| Starting materials | Composition [wt %] | Type/Function |
|---|---|---|
| Ketamine HCl | 39.0 | Active ingredient |
| PVA 4-88 | 31.0 | Polymer |
| PVA 40-88 | 6.0 | Polymer |
| Xylitol | 2.0 | Sweetener and disintegrant |
| Tween 80 | 1.0 | Plasticizer |
| Sorbitol | 9.0 | Sweetener and disintegrant |
| Sucralose | 1.0 | Sweetener, disintegrant, and |
| | | humectant |
| Glycerol | 10.0 | Plasticizer |
| Menthol | 1.0 | Flavor |
| Basis weight | 109.5 g/m² | |
| Solvent | Water | |

### Disintegration Times of the Above Formulations

The disintegration time was determined using method Ph. Eur. 2.9.1. with weight and plate.

| | | |
|---|---|---|
| | Disintegration time | Disintegration time |
| Formulation | measurement 1 | measurement 2 |

| | | | | |
|---|---|---|---|---|
| A1 (reference) | 50 | seconds | 51 | seconds |
| A2 | 4 | seconds | 4 | seconds |
| A3 | 17 | seconds | 18 | seconds |
| A4 | 30 | seconds | 31 | seconds |
| B1 | 8 | seconds | 8 | seconds |
| B2 | 15 | seconds | 15 | seconds |

### Example 2

Oral thin films were prepared with naloxone HCl dihydrate and nalmefene HCL as the active ingredient and their disintegration times were determined according to method Ph. Eur. 2.9.1. with weight and plate.

Formulation C1: PVA foam formulation

| | Composition | | |
|---|---|---|---|
| Starting materials | [wt %] | | Type/Function |
| Naloxone HCl dihydrate | 10 | | Active ingredient |
| PVA 4-88 | 51 | | Polymer |
| PVA 40-88 | 16 | | Polymer |
| Xylitol | 2 | | Sweetener and disintegrant |
| Tween 80 | 1 | | Plasticizer |
| Sorbitol | 9 | | Sweetener and disintegrant |
| Sucralose | 1 | | Sweetener, disintegrant, and |
| | | | humectant |
| Glycerol | 10 | | Plasticizer |
| Basis weight | 128 | g/m² | |
| Solvent | Water | | |
| Disintegration time | 4 | s | |

Formulation C2: PVA film formulation

| | Composition | | |
|---|---|---|---|
| Starting materials | [wt %] | | Type/Function |
| Naloxone HCl dihydrate | 10 | | Active ingredient |
| PVA 4-88 | 56 | | Polymer |
| PVA 40-88 | 6 | | Polymer |
| Xylitol | 2 | | Sweetener and disintegrant |
| Span 83 | 1 | | Emulsifier |
| Tween 80 | 1 | | Plasticizer |
| Sorbitol | 9 | | Sweetener and disintegrant |
| Sucralose | 1 | | Sweetener, disintegrant, and |
| | | | humectant |
| Glycerol | 9 | | Plasticizer |
| Rice starch | 5 | | Disintegrant |
| Basis weight | 147 | g/m² | |
| Solvent | Water | | |
| Disintegration time | 33 | s | |

Formulation C3: PVA foam formulation

| | Composition | | |
|---|---|---|---|
| Starting materials | [wt %] | | Type/Function |
| Naloxone HCl dihydrate | 10 | | Active ingredient |
| PVA 4-88 | 51 | | Polymer |
| PVA 40-88 | 16 | | Polymer |
| Xylitol | 2 | | Sweetener and disintegrant |
| Sodium bisulfite | 0.1 | | Antioxidant |
| Tween 80 | 1 | | Plasticizer |
| Sorbitol | 9 | | Sweetener and disintegrant |
| Sucralose | 1 | | Sweetener, disintegrant, and |
| | | | humectant |
| Glycerol | 9.9 | | Plasticizer |
| Basis weight | 176 | g/m² | |
| Solvent | Water | | |
| Disintegration time | 5 | s | |

Formulation C4: PVA foam formulation; PVA solutions were adjusted to pH 4

| | Composition | | |
|---|---|---|---|
| Starting materials | [wt %] | | Type/Function |
| Naloxone HCl dihydrate | 10 | | Active ingredient |
| PVA 4-88 | 51 | | Polymer |
| PVA 40-88 | 16 | | Polymer |
| Xylitol | 2 | | Sweetener and disintegrant |
| Sodium bisulfite | 0.1 | | Antioxidant |
| Tween 80 | 1 | | Plasticizer |
| Sorbitol | 9 | | Sweetener and disintegrant |
| Sucralose | 1 | | Sweetener, disintegrant, and |
| | | | humectant |
| Glycerol | 9.9 | | Plasticizer |
| Basis weight | 152 | g/m² | |
| Solvent | Water | | |
| Disintegration time | 4 | s | |

Formulation C5: PVA foam formulation; PVA solutions were adjusted to pH 8

| | Composition | | |
|---|---|---|---|
| Starting materials | [wt %] | | Type/Function |
| Naloxone HCl dihydrate | 10 | | Active ingredient |
| PVA 4-88 | 51 | | Polymer |
| PVA 40-88 | 16 | | Polymer |
| Xylitol | 2 | | Sweetener and disintegrant |
| Sodium bisulfite | 0.1 | | Antioxidant |
| Tween 80 | 1 | | Plasticizer |
| Sorbitol | 9 | | Sweetener and disintegrant |
| Sucralose | 1 | | Sweetener, disintegrant, and |
| | | | humectant |
| Glycerol | 9.9 | | Plasticizer |
| Basis weight | 153 | g/m² | |
| Solvent | Water | | |
| Disintegration time | 7 | s | |

Formulation C6: PVA film formulation

| | Composition | | |
|---|---|---|---|
| Starting materials | [wt %] | | Type/Function |
| Naloxone HCl dihydrate | 10 | | Active ingredient |
| PVA 4-88 | 56 | | Polymer |
| PVA 40-88 | 6 | | Polymer |
| Xylitol | 2 | | Sweetener and disintegrant |
| Span 83 | 1 | | Emulsifier |
| Sodium bisulfite | 0.1 | | Antioxidant |
| Tween 80 | 1 | | Plasticizer |
| Sorbitol | 9 | | Sweetener and disintegrant |
| Sucralose | 1 | | Sweetener, disintegrant, and |
| | | | humectant |
| Glycerol | 8.9 | | Plasticizer |
| Rice starch | 5 | | Disintegrant |
| Basis weight | 147 | g/m² | |
| Solvent | Water | | |
| Disintegration time | 20 | s | |

Formulation C7: PVA film formulation; PVA solutions were adjusted to pH 4

| | Composition | | |
|---|---|---|---|
| Starting materials | [wt %] | | Type/Function |
| Naloxone HCl dihydrate | 10 | | Active ingredient |
| PVA 4-88 | 56 | | Polymer |
| PVA 40-88 | 6 | | Polymer |
| Xylitol | 2 | | Sweetener and disintegrant |
| Span 83 | 1 | | Emulsifier |
| Sodium bisulfite | 0.1 | | Antioxidant |
| Tween 80 | 1 | | Plasticizer |
| Sorbitol | 9 | | Sweetener and disintegrant |
| Sucralose | 1 | | Sweetener, disintegrant, and |
| | | | humectant |
| Glycerol | 8.9 | | Plasticizer |
| Rice starch | 5 | | Disintegrant |
| Basis weight | 157 | g/m² | |
| Solvent | Water | | |
| Disintegration time | 22 | s | |

Formulation C8: PVA film formulation; PVA solutions were adjusted to pH 8

| | Composition | | |
|---|---|---|---|
| Starting materials | [wt %] | | Type/Function |
| Naloxone HCl dihydrate | 10 | | Active ingredient |
| PVA 4-88 | 56 | | Polymer |
| PVA 40-88 | 6 | | Polymer |
| Xylitol | 2 | | Sweetener and disintegrant |
| Span 83 | 1 | | Emulsifier |
| Sodium bisulfite | 0.1 | | Antioxidant |
| Tween 80 | 1 | | Plasticizer |
| Sorbitol | 9 | | Sweetener and disintegrant |
| Sucralose | 1 | | Sweetener, disintegrant, and |
| | | | humectant |
| Glycerol | 8.9 | | Plasticizer |
| Rice starch | 5 | | Disintegrant |
| Basis weight | 154 | g/m² | |
| Solvent | Water | | |
| Disintegration time | 28 | s | |

Formulation C9: PVA foam formulation

| Starting materials | Composition [wt %] | | Type/Function |
|---|---|---|---|
| Nalmefene HCl | 10 | | Active ingredient |
| PVA 4-88 | 51 | | Polymer |
| PVA 40-88 | 16 | | Polymer |
| Xylitol | 2 | | Sweetener and disintegrant |
| Tween 80 | 1 | | Plasticizer |
| Sorbitol | 9 | | Sweetener and disintegrant |
| Sucralose | 1 | | Sweetener, disintegrant, and |
| | | | humectant |
| Glycerol | 10 | | Plasticizer |
| Basis weight | 164 | g/m² | |
| Solvent | Water | | |
| Disintegration time | 10 | s | |

### Example 3

Oral thin films were prepared with vortioxetine HBr as the active ingredient and their disintegration times were determined according to method Ph. Eur. 2.9.1. with weight and plate.

Formulation D1: PVA foam formulation

| Starting materials | Composition [wt %] | | Type/Function |
|---|---|---|---|
| Vortioxetine HBr | 30 | | Active ingredient |
| PVA 4-88 | 36 | | Polymer |
| PVA 40-88 | 11 | | Polymer |
| Xylitol | 2 | | Sweetener and disintegrant |
| Sodium bisulfite | | | Antioxidant |
| Tween 80 | 1 | | Plasticizer |
| Sorbitol | 9 | | Sweetener and disintegrant |
| Sucralose | 1 | | Sweetener, disintegrant, and |
| | | | humectant |
| Glycerol | 10 | | Plasticizer |
| Basis weight | 167.5 | g/m² | |
| Solvent | Water | | |
| Disintegration time | 5 | s | |

Formulation D2: PVA film formulation

| Starting materials | Composition [wt %] | | Type/Function |
|---|---|---|---|
| Vortioxetine HBr | 30 | | Active ingredient |
| PVA 4-88 | 37 | | Polymer |
| PVA 40-88 | 4 | | Polymer |
| Xylitol | 2 | | Sweetener and disintegrant |
| Span 83 | 1 | | |
| Tween 80 | 1 | | Plasticizer |
| Sorbitol | 9 | | Sweetener and disintegrant |
| Sucralose | 1 | | Sweetener, disintegrant, and |
| | | | humectant |
| Glycerol | 9 | | Plasticizer |
| Rice starch | 6 | | Disintegrant |
| Basis weight | 153.6 | g/m² | |
| Solvent | Water | | |
| Disintegration time | 19 | s | |

Formulation D3: PVA film formulation

| Starting materials | Composition [wt %] | | Type/Function |
|---|---|---|---|
| Vortioxetine HBr | 30 | | Active ingredient |
| PVA 4-88 | 36 | | Polymer |
| PVA 40-88 | 11 | | Polymer |
| Xylitol | 2 | | Sweetener and disintegrant |
| Tween 80 | 1 | | Plasticizer |
| Sorbitol | 9 | | Sweetener and disintegrant |
| Sucralose | 1 | | Sweetener, disintegrant, and |
| | | | humectant |
| Glycerol | 7 | | Plasticizer |
| Rice starch | 3 | | Disintegrant |
| Basis weight | 148.9 | g/m² | |
| Solvent | Water | | |
| Disintegration time | 4 | s | |

For the above formulations D1 to D3, the in-vitro permeation was carried out using Franz diffusion cells (volume 10 mL) at 37° C. At predetermined changeover times, the acceptor medium used in each case was completely replaced by a new one, and the content of permeated active ingredient in these acceptor solutions was determined using HPLC.

Phosphate buffer pH 7.4 was used as the acceptor medium.

Dermatomized skin from the esophagus of a pig with a layer thickness of 400 μm was used as a skin model.

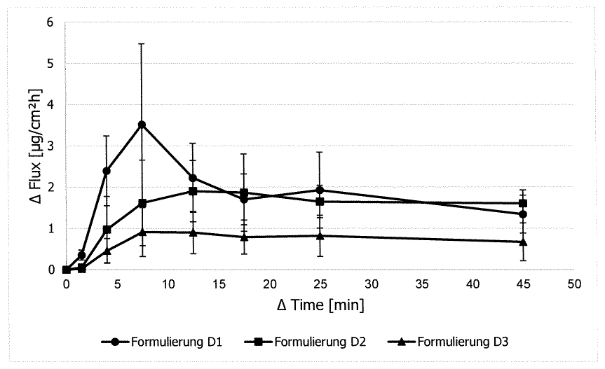

The results are summarized in FIG. 1.

1. An oral thin film comprising at least one matrix layer, wherein the at least one matrix layer comprises at least one polyvinyl alcohol having a lower average molecular weight of 10,000 to about 75,000 g/mol and at least one polyvinyl alcohol having a higher average molecular weight of 140,000 to 300,000 g/mol and at least one active pharmaceutical ingredient, wherein said at least one polyvinyl alcohol having a lower average molecular weight and said at least one polyvinyl alcohol having a higher average molecular weight are present in the matrix layer in a total amount of 15 to 70 wt % based on the total weight of the matrix layer, and the weight ratio of said at least one polyvinyl alcohol having a lower average molecular weight to said at least one polyvinyl alcohol having a higher average molecular weight is in the range from 20:1 to 2:1.

2. The oral thin film according to claim 1, wherein the at least one polyvinyl alcohol having a lower average molecular weight has an average molecular weight in the range of 10,000 to about 40,000 g/mol and/or the at least one polyvinyl alcohol having a higher average molecular weight has an average molecular weight in the range of 140,000 to 220,000 g/mol.

3. The oral thin film according to claim 1, wherein the at least one polyvinyl alcohol having a lower average molecular weight and the at least one polyvinyl alcohol having a higher average molecular weight are present in the matrix layer in a total amount of from 20 to 55 wt % based on the total weight of the matrix layer.

4. The oral thin film according to claim 1, wherein the weight ratio of the at least one polyvinyl alcohol having a lower average molecular weight to the at least one polyvinyl alcohol having a higher average molecular weight is in the range from 15:1 to 3.

5. The oral thin film according to claim 1, wherein the amount of the at least one polyvinyl alcohol having a higher average molecular weight in the matrix layer is in the range from 1 to 18 wt % based on the total weight of the matrix layer.

6. The oral thin film according to claim 1, wherein the at least one polyvinyl alcohol having a lower average molecular weight is polyvinyl alcohol 4-88 and/or the at least one polyvinyl alcohol having a higher average molecular weight is polyvinyl alcohol 40-88.

7. The oral thin film according to claim 1, wherein the matrix layer further comprises at least one hygroscopic additive having more than 3 carbon atoms, preferably selected from sugars, sugar alcohols, starches and starch derivatives, or a mixture thereof.

8. The oral thin film according to claim 7, wherein the at least one hygroscopic additive having more than 3 carbon atoms is selected from sorbitol, mannitol, isomalt, lactitol, sorbitol (glucitol) and xylitol, threitol, erythritol, starches, including starch derivatives, sucralose, or a mixture of two or more of these compounds.

9. The oral thin film according to claim 7, wherein the at least one hygroscopic additive having more than 3 carbon atoms is present in an amount of 5 to 20 wt %, based on the total weight of the matrix layer.

10. The oral thin film according to claim 1, wherein the at least one active pharmaceutical ingredient comprises or is ulipristal acetate or ketamine or naloxone, or nalmefene, or vortioxetine, or a respective pharmaceutically acceptable salt thereof.

11. The oral thin film according to claim 1, wherein the at least one active pharmaceutical ingredient or a pharmaceutically acceptable salt thereof is present in the matrix layer in an amount of 10 to 60 wt % based on the total weight of the matrix layer.

12. The oral thin film according to claim 1, wherein the matrix layer further comprises at least one excipient selected from the group consisting of colorants, flavorings, sweeteners, plasticizers, taste-masking agents, emulsifiers, enhancers, disintegrants, solubilizers, pH regulators, preservatives and/or antioxidants.

13. The oral thin film according to claim 1, wherein the matrix layer is a foamed matrix layer.

14. The oral thin film according to claim 1, wherein the basis weight of the matrix layer is 50 to 220 g/m².

15. A method for the treatment or prevention of pain and/or depression or as an anesthetic and/or in the treatment of opiate overdose comprising administrating the oral thin film of claim 1.

16. The oral thin film of claim 1, wherein the at least one polyvinyl alcohol having a lower average molecular weight has an average molecular weight in the range of 25,000 to about 35,000 g/mol and/or the at least one polyvinyl alcohol having a higher average molecular weight has an average molecular weight in the range of 200,000 to 210,000 g/mol.

17. The oral thin film according to claim 1, wherein the weight ratio of the at least one polyvinyl alcohol having a lower average molecular weight to the at least one polyvinyl alcohol having a higher average molecular weight is in the range from 9:1 to 7:1.

18. The oral thin film according to claim 1, wherein the amount of the at least one polyvinyl alcohol having a higher average molecular weight in the matrix layer is in the range from 3 to 4 wt % based on the total weight of the matrix layer.

19. The oral thin film according to claim 1, wherein the at least one polyvinyl alcohol having a lower average molecular weight and the at least one polyvinyl alcohol having a higher average molecular weight are present in the matrix layer in a total amount of from 25 to 40 wt % based on the total weight of the matrix layer;
the at least one hygroscopic additive having more than 3 carbon atoms is present in an amount of 5 to 15 wt %, based on the total weight of the matrix layer;
the at least one active pharmaceutical ingredient or a pharmaceutically acceptable salt thereof is present in the matrix layer in an amount of 30 to 50 wt % based on the total weight of the matrix layer; and
the basis weight of the matrix layer is 70 to 130 g/m².

20. The oral thin film according to claim 1, wherein the at least one active pharmaceutical ingredient comprises ulipristal acetate or ketamine; and
the matrix layer further comprises one or more glycerol plasticizers.
